(19) United States
(12) Patent Application Publication
(10) Pub. No.: US 2005/0025743 A1
(43) Pub. Date: Feb. 3, 2005

(54) Method of preventing adhesions wtih ifn-upsilon

(76) Inventors: Michael P. Diamond, Grosse Pointe, MI (US); Ghassan M Saed, West Bloomfield, MI (US)

Correspondence Address: ROPES & GRAY LLP, ONE INTERNATIONAL PLACE, BOSTON, MA, 02110-2624, US

(21) Appl. No.: 10/471,892

(22) PCT Filed: Mar. 8, 2002
(86) PCT No.: PCT/US02/07200

(30) Foreign Application Priority Data
Mar. 13, 2001 (US) 60275381

(51) Int. Cl. A61K 38/21; A61K 38/39

(52) U.S. Cl. 424/85.6; 514/21

(57) ABSTRACT

Methods for the prevention of adhesion formation and development involve the administration of therapeutic formulations to a patient which include, as active ingredients, IFN-γ or IFN-γ enhancers. The IFN-γ or IFN-γ enhancers are preferably administered to fibrosis tissues in a subject prior to an event which induces adhesion formation, such as a surgical event.

### BACKGROUND OF THE INVENTION

It is well established that injuries to the peritoneal surface of the peritoneum result in the development of post-operative adhesions in the vast majority of patients following surgery. The peritoneum is composed of mesothelial cells with submesothelial tissue containing fibroblasts, macrophages and blood vessels.

Cellular processes resulting in either normal peritoneal tissue repair or the development of adhesions include migration, proliferation and/or differentiation of several cell types, among them inflammatory, immune, mesothelial and fibroblast cells. Molecules produced locally by these cells regulate fibrinolytic activity, tissue remodeling and angiogenesis, as well as the synthesis and deposition of extracellular matrix material (ECM), and these processes are central to the development of adhesions.

The molecular events underlying peritoneal wound healing and the development of fibrous adhesions are complex, multifactoral and not well defined. The cascade of events that leads to peritoneal wound repair in many aspects resembles the events that occur during skin wound healing, which is characterized by inflammation, cellular migration, proliferation, phenotypic differentiation and tissue remodeling. During wound healing, fibroblasts invade the wound in the first few days of healing, and these fibroblasts have multiple functions important to wound repair. These functions include collagen synthesis, ECM reorganization, and wound contraction, resulting in mature scar formation.

Tissue remodeling involves the deposition and degradation of the extracellular matrix, which is a highly regulated process occurring during wound repair, and which is influenced by a host of locally expressed growth factors, cytokines and eicosanoids. The extracellular matrix is a dynamic component capable of modulating various cellular activities including cell-cell interaction, proliferation, differentiation and sequestration of potent biological response modifiers from the wound environment. In addition, excess production and deposition of the extracellular matrix is a key factor in producing tissue fibrosis throughout the body, including the development of peritoneal adhesions.

Apoptosis, or programmed cell death, is a component of normal development and differentiation in most tissues, including tissues involved in adhesion development. Apoptosis is a complex process occurring in a wide variety of organisms that removes aging or injured cells from the body. This type of cell death may be inhibited by deleterious stimuli, such as hypoxia, distorting the balance of cellular proliferation, differentiation and death, thereby impairing the normal peritoneal wound repair process. Indeed, a lower degree of apoptosis is found in dermal fibroblasts isolated from the skin of keloids and hypertrophic scars, as compared to normal skin fibroblasts.

There are at least two signaling pathways which are known to trigger apoptosis. The first is mediated by the interaction of membrane receptors and ligands, such as Fas ligand and TNF-α. The second pathway is triggered by exogenous stimuli such as hypoxia, radiation and chemotherapeutic drugs, wherein the death signal is transmitted through the mitochondria. This second pathway involves the altered expression of p53 and members of the Bcl-2 family. p53 point mutations have been detected in fibroblasts isolated from keloids, and the proapoptotic protein Bax which is expressed primarily in involved skin fibroblasts. In contrast to Bax, the antiapoptotic protein Bcl-2 has been found to be expressed at higher levels in fibroblast cells.

Hypoxia has a variety of effects on fibroblasts, both in vivo and in vitro. Hypoxia stimulates matrix synthesis with increased expression of fibronectin, and type I and type III collagens. Hypoxia also stimulates the production of a variety of growth factors, including the profibrotic factor transforming growth factor-β1 (TGF-β1) in human mesothelial and fibroblast cells. Although fibroblasts are invariably exposed to hypoxia in ischemic conditions, the effect of hypoxia on the apoptosis of human peritoneal and adhesion fibroblasts is not known.

The overexpression of TGF-β1 has been implicated in fibrotic disorders at various sites throughout the body, such as pulmonary fibrosis, glomerulonephritis, cirrhosis of the liver, and dermal scarring. Elevated levels of TGF-β expression occurs in adhesion tissues, in the peritoneal fluid of patients with adhesions, and in surgically induced adhesion formation in animal models. Mice that are heterozygous for TGF-β1 (+/−) have been shown to have significantly lower adhesions, and express at least two fold lower TGF-β1 protein in their peritoneal fluids, as compared with wild type (+/+) animals as early as 2 hours post-injury. See Krause et al., J. Invest Surg., 12, pages 31-38 (1999). Additionally, the postoperative peritoneal administration of TGF-β1 has been shown to increase the incidence of adhesion formation, while neutralizing antibodies directed against TGF-β reduce such incidence. Lucas et al., J. Surg. Res., 65, pages 135-138 (1996); and Williams et al., J. Surg. Res., 52, pages 65-70 (1991).

It has been suggested that peritoneal adhesions develop in the vast majority of subjects, with more frequent occurrence in certain subjects following surgical procedures as opposed to others. The molecular basis for such predisposition is not known. Accordingly, it is an objective of this invention to provide a method for preventing or reducing the incidence of post-operative surgical adhesions with or without the use of barrier materials by addressing the molecular basis of the condition.

### SUMMARY OF THE INVENTION

It has now been discovered that the apoptosis rate is significantly higher in human normal peritoneal fibroblasts than in adhesion fibroblasts, and that hypoxia inhibits apoptosis and enhances the proliferation of adhesion fibroblasts in vivo. These discoveries have led to the development of novel methods for treating surgical adhesions.

In one aspect, the invention comprises a method for the prevention or remediation of surgical adhesions by treating a patient at risk of developing such adhesions with a therapeutic formulation that increases apoptosis of adhesion fibroblasts in vivo. Preferably, the formulations of this invention comprise IFN-γ or IFN-γ enhancers. The formulations of this invention can be applied topically, to the site of potential adhesion formation, or systemically, to increase the level of IFN-γ in tissues and cells which may be predisposed to form adhesions.

While not wishing to be bound by any particular theory, it is believed that the IFN-γ may assist in preventing the overexpression and accumulation of the extracellular matrix (ECM) which is essential to peritoneal adhesion formation following surgically-induced tissue trauma. Furthermore, the IFN-γ may also assist in manipulating the ratio of active TGF-β isoforms in the extracellular matrix, and in particular, in reducing the TGF-β1/TGF-β2 ratio, or preventing activation of TGF-β isoforms. This is believed to result in a reduction in scarring and fibrosis by controlling the chemotactic recruitment of fibroblasts and inflammatory cells, by regulating angiogenesis, and by regulating the synthesis, deposition and turnover of components of the extracellular matrix.

The term “IFN-γ”, as used herein, means gamma interferon, and any substances and molecules which have the biological characteristics of gamma interferon. An “IFN-γ enhancer”, as used herein, is a biological or chemical agent which increases the expression levels of IFN-γ in cells, such as peritoneal fibroblast cells.

An additional aspect of this invention involves a method for the detection of a predisposition in a subject to adhesion formation which comprises the detection of elevated levels of type I collagen, but not fibronectin, in tissues in a subject. This higher level of expression of type I collagen is believed to indicate a predisposition to develop adhesions in the affected cells and tissue. Once detected, this predisposition for adhesion formation can then be treated using the treatment procedures of this invention.

### BEST MODES FOR CARRYING OUT THE INVENTION

### DETAILED DESCRIPTION OF THE INVENTION

Peritoneal mesothelial cells that line the serosal surface of the peritoneal cavity provide a natural protective barrier that prevents the organs from adhering to adjacent opposing surfaces. However, cellular or tissue injury that is induced following a surgical procedure, an infection or inflammation compromises the integrity of the mesothelial cells, and can result in a local biological response with the objective of repairing the defected surface. If a cellular or tissue injury is relatively extensive, it can lead to excess migration and proliferation of various wound cells, such as fibroblasts. This response initiates a cascade of events that often result in the development of peritoneal adhesions, known to be the major cause of bowel obstruction, pain and infertility, and hospital readmissions.

It has now been found that IFN-γ acts like an antifibrogenic cytokine that can be used as a therapeutic agent to reduce or prevent postoperative adhesions and tissue fibrosis. IFN-γ can block the stimulating effect of hypoxia caused by severe tissue damage on type I collagen and fibronectin expression. This, in turn, prevents the overexpression and accumulation of the extracellular matrix which is central to peritoneal adhesion formation following surgically-induced tissue trauma.

It has also been found, according to the invention, that the administration of the preparations of the invention to tissue and organs at the site of potential adhesion formation, locally or systemically, can induce protection against postoperative surgical adhesion development. The preparations of the invention are useful for treating or preventing adhesions that form at a site and that have potential or actual deleterious biological effects. These preparations include both IFN-γ and IFN-γ enhancers.

Adhesions that can be successfully treated according to the method of this invention include, but are not limited to, primary and secondary adhesions in the following sites in a subject: in the abdominal cavity, including intestine to intestine, and intestine to peritoneum; in the pelvic cavity, including adhesion of the uterus, ovaries or fallopian tubes to other structures including each other and the pelvic wall; in tendons and their support structures, including tendon to synovium; in the repair of nerve sheaths; in the repair of the spinal column or disks; in the pericardium; in the treatment of joints for inflammation, and to prevent pannus formation; in the extraoccular muscle, to prevent adhesions from limiting the field of vision; and in any situation or location in which adhesions form that can impair function or cause pain.

The prevention of postoperative surgical adhesion development in a subject includes prophylactic treatment to prevent adhesion development following planned or elective surgical procedures, as well as following emergency operations. In addition to the surgical procedures described above, elective surgeries within the scope of this invention include the following intraabdominal surgeries: right hemicolectomy; left hemicolectomy; myomectomy; ovarian cystectomy; hysterectomy; oophorectomy; salpingectomy; endometriosis; ectopic pregnancy, bladder suspension; vaginal suspension; gastrectomy; pancreatectomy; sigmoid colectomy; subtotal colectomy; total colectomy; laparoscopic or open cholecystectomy; gastrectomy; pancreatectomy; splenectomy; liver, pancreas, small bowel, or kidney transplantation; lysis of adhesions; cesarean sections and other pelvic procedures, uterine surgery, etc. Emergency intraabdominal surgeries include those surgeries used to correct the following conditions: perforated ulcer (duodenal or gastric); perforated diverticulitis; opthamology; obstructive diverticulitis; bowel obstruction; perforated appendicitis; blunt abdominal trauma; penetrating abdominal trauma; ruptured abdominal aortic aneurysm, cardiac surgeries, open and endoscopic orthopedic surgeries, neurosurgeries, gynecologic and pelvic surgeries, and surgeries to correct wound infections.

The preparations of the invention are administered to a subject in an effective amount for inducing protection against postoperative surgical adhesion development. An “effective amount” for inducing protection against postoperative surgical adhesion development, as used herein, is that amount of pharmaceutical composition that will, either alone or together with further doses or additional therapeutic compounds, inhibit or prevent the development of postoperative surgical adhesions.

The term “subject,” as used herein, means a human or non-human mammal, including but not limited to, a dog, cat, horse, cow, pig, sheep, goat, chicken, primate, rat, and mouse.

The terms “prevent” and “preventing” as used herein refer to inhibiting completely or partially a biological response, as well as inhibiting an increase in a biological response. For instance, prevention of adhesion-development refers to partially or completely inhibiting adhesion formation and adhesion reformation, as well as inhibiting an increase in adhesion formation and adhesion reformation. The preparations of the invention when administered “in conjunction with” a surgical procedure, are administered close enough in time with the surgery or trauma that predispose the host to adhesion development, so that a protective effect against the particular disorder is obtained. The preparations may be administered long before the surgery, e.g., in the case of elective surgery (i.e., weeks or even months), preferably with booster administrations closer in time to (and even after) the surgery. Particularly in emergency situations, the preparations may be administered immediately before (minutes to hours), during and/or after the surgery. It is important only that the preparation be administered close enough in time so as to enhance the subject's response against adhesions, thereby increasing the chances of a successful host response and reducing the likelihood of adhesion development. The present invention provides pharmaceutical compositions for medical use, which in some aspects comprise the preparations of the invention together with one or more pharmaceutically acceptable carriers and optionally other therapeutic ingredients. Acceptable carriers include mechanical or physical barrier materials, such as hyaluronic acid and carboxymethylcellulose. Thus the invention may also include pharmaceutical compositions in combination with an anti-infectious agent such as an antibacterial or anti-viral agent, an anti-inflammatory agent, an antibiotic, or other therapeutic agent, and a pharmaceutically acceptable carrier. The pharmaceutical compositions useful in the invention may be delivered separately with the other therapeutic agents, or in the form of therapeutic cocktails. A therapeutic cocktail includes a mixture of the pharmaceutical composition of the invention and another therapeutic agent. In this embodiment, a common administration vehicle (e.g., tablet, implant, injectable solution, etc.) contains both the pharmaceutical composition and another therapeutic agent. Alternatively, the other therapeutic agent can be separately dosed if desired.

The precise amount of the therapeutic agent used in combination with the pharmaceutical compositions of the invention depends upon a variety of factors, including the particular pharmaceutical composition selected, the dose and dose-timing selected, the mode of administration, the nature of any surgical or medical procedure contemplated, and the characteristics of the subject. Where local administration is carried out, it will be understood that very small amounts may be required (nanograms and possibly picograms). The precise amounts selected can be determined without undue experimentation, particularly since a threshold amount is any amount which will favorably enhance the response.

Multiple doses of the pharmaceutical compositions of the invention are contemplated. For instance, when being administered in conjunction with a surgical procedure, the compositions of the invention can be administered in multiple doses over a three week to one day period preceding surgery. Further, doses may be administered post surgery as well. Any regimen that prevents or retards the development of adhesions may be used, although optimum doses and dosing regimens are those that would not only inhibit the development of adhesion formation, but also would result in complete protection against adhesion development. Desired time intervals for the delivery of multiple doses of a particular pharmaceutical composition can be determined by one of ordinary skill in the art employing no more than routine experimentation.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Suitable buffering agents include: acetic acid or its salt (1-2% w/v); citric acid or its salt (1-3% w/v); boric acid or its salt (0.5-2.5% w/v); succinic acid; and phosphoric acid or its salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a pharmaceutical composition optionally included in a pharmaceutically acceptable carrier. The term “pharmaceutically acceptable carrier” means one or more compatible solid or liquid filler, and dilutants or encapsulating substances which are suitable for administration to a human or other animal. The term “carrier” denotes an organic or inorganic ingredient, natural or synthetic, with which the active ingredient is combined to facilitate the application of the composition. The components of the pharmaceutical compositions also are capable of being commingled with the pharmaceutical compositions of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid find use in the preparation of injectables. Carrier formulations suitable for subcutaneous, intramuscular, intraperitoneal or intravenous administrations may be found in Remington's Pharmaceutical Sciences, Mack Publishing Company, Easton, Pa.

The pharmaceutical compositions useful in the invention may be delivered in mixtures of more than one pharmaceutical composition. A mixture may consist of several pharmaceutical compositions.

A variety of administration routes are available. The particular mode selected will depend, of course, upon the particular pharmaceutical composition selected, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration include parenteral, injection, infusion, deposition, implantation, anal or vaginal supposition, oral ingestion, inhalation, and topical administration. Injections can be intravenous, intradermal, subcutaneous, intramuscular, or interperitoneal. For example, the pharmaceutical composition can be injected directly into the surgical site for the prevention of adhesions. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the pharmaceutical composition with an aerosol in an inhaler, either alone or attached to a carrier that can be absorbed. For systemic administration, it may be preferred that the pharmaceutical composition is encapsulated in liposomes. The term “parenteral” includes subcutaneous injections, intravenous, intramuscular, intraperitoneal, intrasternal injection or infusion techniques.

In certain preferred embodiments of the invention, the administration can be designed so as to result in sequential exposure of the pharmaceutical composition over some period of time, e.g., hours, days, weeks, months or years. This can be accomplished by repeated administrations of the pharmaceutical composition, by one of the methods described above, or alternatively, by a sustained-release delivery system in which the pharmaceutical composition is delivered to the subject for a prolonged period without repeated administrations. By sustained-release delivery system, it is meant that total release of the pharmaceutical composition does not occur immediately upon administration, but rather is delayed for some period of time. Release can occur in bursts or it can occur gradually and continuously. Administration of such a system can be, e.g., by long-lasting oral dosage forms, bolus injections, transdermal patches, and subcutaneous implants.

Examples of systems in which release occurs in bursts includes, e.g., systems in which the pharmaceutical composition is entrapped in liposomes which are encapsulated in a polymer matrix, the liposomes being sensitive to specific stimuli, e.g., temperature, pH, light or a degrading enzyme and systems in which the pharmaceutical composition is encapsulated by an ionically-coated microcapsule with a microcapsule core degrading enzyme. Examples of systems in which release of the pharmaceutical composition is gradual and continuous include, e.g., erosional systems in which the pharmaceutical composition is contained in a form within a matrix and effusional systems in which the pharmaceutical composition permeates at a controlled rate, e.g., through a polymer. Such sustained release systems can be e.g., in the form of pellets, or capsules.

In one particular embodiment, the preferred sustained release device is a biocompatible microparticle or microencapsulated product or implant that is suitable for implantation or administration to the mammalian recipient. Exemplary bioerodible implants that are useful in accordance with this method are described in PCT International application no. PCT/US/03307 (Publication No. WO 95/24929, entitled “Polymeric Gene Delivery System.” The polymeric matrix preferably is in the form of a microparticle such as a microsphere (wherein the pharmaceutical composition is dispersed throughout a solid polymeric matrix) or a microcapsule (wherein the pharmaceutical composition is stored in the core of a polymeric shell). Other forms of the polymeric matrix for containing the pharmaceutical composition include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue. The polymeric matrix composition can be selected to have both favorable degradation rates and also to be formed of a material which is bioadhesive, to further increase the effectiveness of transfer when the matrix is administered to a mucosal surface. The matrix composition also can be selected not to degrade, but rather to release by diffusion over an extended period of time. The biocompatible microsphere may be suitable for oral delivery. Such microspheres are disclosed in Chickering et al., Biotech. And Bioeng., (1996) 52:96-101 and Mathiowitz et al., Nature, (1997) 386:410-414 and PCT Patent Application WO 97/03702.

Both non-biodegradable and biodegradable polymeric matrices can be used to deliver the pharmaceutical compositions to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water, and further optionally is cross-linked with multi-valentions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, casein, gelatin, glutin, polyanhydrides, polyacrylic acid, alginate, chitosan, poly(methyl methacrylates), poly(ethyl methacrylates), poly(butylmethacrylate), polyhyaluronic acids, poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Other sustained release delivery systems useful according to the invention include, but are not limited to, fatty acids and a medicinal pump. Preferably the fatty acids are C₉-C₂₀ fatty acids.

The compositions may conveniently be presented in unit dosage form and may be prepared by any of the methods well known in the art of pharmacy. Such methods include the step of bringing the pharmaceutical composition into association with a carrier which constitutes one or more accessory ingredients. In general, the compositions are prepared by uniformly and intimately bringing the pharmaceutical composition into association with a liquid carrier, a finely divided solid carrier, or both, and then, if necessary, shaping the product. The pharmaceutical composition may be stored in a lyophilized condition.

The pharmaceutical compositions can be suspended in a liquid, e.g., in dissolved form or colloidal form. The liquid can be a solvent, partial solvent, or non-solvent. In many cases, water or an organic liquid can be used.

The pharmaceutical compositions are administered to the subject in a therapeutically-effective amount. By therapeutically-effective amount is meant that amount which is capable of at least partially preventing, reversing, reducing, decreasing, ameliorating, or otherwise suppressing adhesions. A therapeutically-effective amount can be determined on an individual basis and is based, at least in part, on consideration of the age, sex, size, and health of the subject; the type of pharmaceutical composition used, the type of delivery system used; the time of administration; and whether a single, multiple, or controlled-release dose regimen is employed. A therapeutically-effective amount can be determined by one of ordinary skill in the art employing such factors and using no more than routine experimentation.

The dosage concentration of the pharmaceutical composition actually administered is dependent, at least in part, upon the final concentration of pharmaceutical composition that is desired at the site of action, the method of administration, the efficacy of the particular pharmaceutical composition, the longevity of the particular pharmaceutical composition, and the timing of administration. Preferably, the dosage form is such that it does not substantially deleteriously effect the subject. The dosage can be determined by one of ordinary skill in the art employing such factors and using no more than routine experimentation.

The following examples serve to illustrate the invention without limiting it thereby. It will be understood that variations and modifications can be made without departing from the spirit and scope of the invention.

### EXAMPLE 1

The purpose of this example is to determine the effect of IFN-γ treatment of normal human peritoneal and adhesion fibroblasts under normal and hypoxic conditions. In particular, the expression of type I collagen and fibronectin in these fibroblast cells in response to IFN-γ treatment is evaluated.

Primary cultures of normal peritoneum human fibroblasts and adhesion fibroblasts are treated with IFN-γ under both normal and hypoxic conditions. For purposes of this example, a normal condition means a 20% O₂ environment, and a hypoxic condition means a 2% O₂ environment. The total RNA is extracted from each treatment and subjected to multiplex RT/PCR analysis to quantitate the relative change in mRNA levels of type I collagen and fibronectin.

The IFN-γ treatment results in a 30% decrease in type I collagen and a 15% decrease in fibronectin mRNA levels, in both adhesion fibroblasts and normal peritoneum fibroblasts. Hypoxia treatment results in a 42% increase in type I collagen mRNA levels in normal peritoneum cells, and a 31% increase in type I collagen mRNA levels in adhesion cells. Hypoxia treatment also results in a 30% increase in fibronectin in both normal peritoneum cells and adhesion cells.

Treatment of cells using both IFN-γ and hypoxia conditions results in no net change in the mRNA levels of type I collagen and fibronectin in both normal peritoneum cells and adhesion cells. It is also found that adhesion fibroblasts have an increased basal level of type I collagen, but not fibronectin, as compared to normal peritoneum cells.

These results show that IFN-γ can block the stimulating effect of hypoxia on type I collagen and fibronectin expression. This supports the view that IFN-γ is antifibrogenic in nature.

### EXAMPLE 2

The purpose of this example is to determine the relative change in the mRNA level of the TGF-β isoforms, TGF-β1 and TGF-β2, in normal peritoneum and adhesion tissues in response to IFN-γ treatment. Normal human peritoneum tissue and adhesion tissue are obtained from the same patient, and the IFN-γ treatment is under both normal and hypoxic conditions.

Primary cultures of normal peritoneum human fibroblasts and adhesion fibroblasts are treated with IFN-γ under both normal and hypoxic conditions. For purposes of this example, a normal condition means a 20% O₂ environment and a hypoxic condition means a 2% O₂ environment. The total RNA is extracted from each treatment and subjected to multiplex RT/PCR analysis to quantitate the relative change in mRNA levels of TGF-β1 and TGF-β2.

The IFN-γ treatment of normal peritoneum cells results in a 22% decrease in TGF-β1 mRNA levels, and a 30% increase in TGF-β2 mRNA levels, which corresponds to a 40% decrease in the TGF-β1/TGF-β2 ratio. In contrast, IFN-γ treatment of adhesion cells results in a 17% decrease in TGF-β1 mRNA levels, and a 22% decrease in TGF-β2 mRNA levels, which corresponds to no significant change in the TGF-β1/TGF-β2 ratio.

Hypoxia treatment of normal peritoneum cells results in a 44% increase in TGF-β1 mRNA levels, and a 24% increase in TGF-β2 mRNA levels, which corresponds to a 16% increase in the TGF-β1/TGF-β2 ratio. In contrast, hypoxia treatment of adhesion cells results in a 16% increase in TGF-β1 mRNA levels, and a 29% decrease in TGF-β2 mRNA levels, which corresponds to a 67% increase in the TGF-β1/TGF-β2 ratio.

These results show that IFN-γ reduces the TGF-β1/TGF-β2 ratio, and also blocks the increase in this ratio due to hypoxia in normal peritoneal fibroblasts but not in adhesion fibroblasts. This differential effect may account for the difference in propensity for adhesion development at sites which have had prior adhesions, and sites with no such prior history.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

1. A method for the prevent or remediation of surgical adhesions comprising treating a patient at risk of developing such adhesions with a therapeutic formulation containing an active ingredient selected from the group consisting of IFN-γ and IFN-γ enhancers.

2. The method of claim 1 wherein the therapeutic formulation is locally administered to the site of potential adhesion formation.

3. The method of claim 1 wherein the administration of the therapeutic formulation results in an increase in apoptosis in adhesion fibroblasts.

4. A method for the detection of a predisposition in a subject to adhesion formation which comprises the detection of elevated levels of type I collagen in the cells or tissue of a subject.

5. The method of claim 4 wherein the cells are fibroblast cells.
